(19) United States
(12) Patent Application Publication
(10) Pub. No.: US 2012/0220648 A1
(43) Pub. Date: Aug. 30, 2012

(54) Method of Treating Aromatic L-Amino Acid Decarboxylase (AADC) Deficiency Using Adeno-Associated Virus (AAV)-AADC Vector

(75) Inventors: Wuh-Liang Hwu, Taipei City (TW); Sheng-Hong Tseng, Taipei City (TW)

(73) Assignee: NATIONAL TAIWAN UNIVERSITY HOSPITAL, Taipei city (TW)

(21) Appl. No.: 13/033,933

(22) Filed: Feb. 24, 2011

(51) Int. Cl. A61K 31/7088; A61P 43/00

(52) U.S. Cl. 514/44 R

(57) ABSTRACT

An adeno-associated virus (AAV) vector is used for treating aromatic L-amino acid decarboxylase (AADC) deficiency. The AAV vector is directly injected into human brain through a stereotactic technique. Activity of AADV is thus recovered for solving the problem of movement disorder.

### TECHNICAL FIELD OF THE DISCLOSURE

The present disclosure relates to treating aromatic L-amino acid decarboxylase (AADC) deficiency; more particularly, relates to using an adeno-associated virus (AAV) viral vector to transfer AADC gene by being directly injected into human brain through a stereotactic technique for solving the problem of movement disorder by recovering activity of AADV.

### DESCRIPTION OF THE RELATED ARTS

Aromatic L-amino acid decarboxylase (AADC) deficiency is a disease where, owing to an inborn gene defect, AADC is not synthesized to function as it normally does. AADC is usually found in striatum. L-dopa and 5-hydroxy-tryptophan are used to synthesize dopamine and serotonin through decarboxylation. Dopamine can be further transformed into epinephrine and norepinephrine through monoamine oxidase. AADC is in charge of synthesizing dopamine in striatum. If AADC is in short, dopamine is not synthesized and may further cause Parkinson's disease with movement disorder.

AADC deficiency was found by Hyland and Clayton in 1990. By analyzing cerebrospinal fluid in children having neurological disease, values of metabolite of neurotransmitter are found abnormal. Most children having AADC deficiency is weak in suction power, difficult in swallowing, easy to be drowsy, and low in body temperature and muscle tone, while they may be easily misdiagnosed as having convulsion or cerebral palsy. After a few months, some clinical symptoms become obvious to those children, like oculogyric crisis, weak muscle tone of trunk, high muscle tone of limb, involuntary movement, easy anger, emotion instability, strong deep tendon reflex, sleep dysfunction or strong reaction to shock. Hence, typical clinical symptoms of AADC deficiency include intermittent oculogyric crisis, weak muscle tone, choreoathetosis in whole body and few autonomic movements. Besides, AADC deficiency may be abided with autonomic dysfunction, which includes spontaneous cold sweating, unstable temperature, nasal congestion, dysfunction in blood pressure control and cardiopulmonary arrest owing to slow heart beating; and, regarding to affects to gastrointestinal tract, includes gastro-esophageal reflux, constipation and diarrhea. Through studying image of neural system, not much hurt is found in the children's brains. Yet, with dopa labeled with radio isotope F18 for positron emission tomography (PET), brains are observed and the labeled isotope is not found in the brains.

For treating AADC deficiency, dopamine receptor agonist can be used to improve movement function. But, after treating with drug, some children get better while some other children do not and get side effect of drug like dyskinesia. Therefore, those children usually die at 5 to 6 years old owing to dysfunction in movement.

AADC is in charge of the final step of synthesizing the neurotransmitters of dopamine and serotonin. Owing to inborn gene defect, patients having hereditary disease of AADC deficiency can not synthesize AADC having normal function and thus have serious movement disorder, which makes those children die at 5 to 6 years old owing to dysfunction in movement. However, there is no effective treatment for AADC deficiency. Although gene supplementary therapy can be applied, AAV-AADC is only used in experiment for Perkin's disease. Hence, the prior arts do not fulfill all users' requests on actual use.

### SUMMARY OF THE DISCLOSURE

The main purpose of the present disclosure is to directly inject an AAV viral vector into human brain through a stereotactic technique for treating AADC deficiency.

To achieve the above purpose, the present disclosure is a method of treating AADC deficiency using AAV-AADC vector, comprising steps of: (a) building a viral vector of serotype 2 of recombinant AAV (rAAV2)-human AADC (hAADC), where the viral vector is obtained from hAADC between cytomegalovirus (CMV) promoter at beginning and SV40 poly A at end; where the viral vector has an adenovirus DNA structure including two inverted terminal repeats (ITR) at two ends of serotype 2 of AAV (AAV2); where the adenovirus DNA structure comprises CMV promoter, β-globin intron, hAADC and SV40 poly A sequentially between the two ITRs; and where the ITR has an antibiotic resistance gene at outboard; and (b) directly injecting the viral vector of rAAV2-hAADC into a brain by using a stereotactic technique. Accordingly, a novel method of treating AADC deficiency using AAV-AADC vector is obtained.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the structural view showing the expression of AADC according to the present disclosure; and

FIG. 2 is the view showing the injection of the viral vector.

### DESCRIPTION OF THE PREFERRED EMBODIMENT

The following description of the preferred embodiment is provided to understand the features and the structures of the present disclosure.

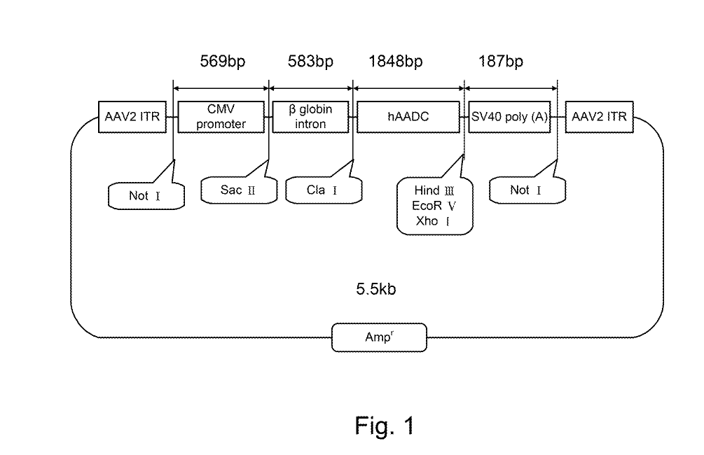

Please refer to FIG. 1, which is a structural view showing an expression of AADC according to the present disclosure. As shown in the figure, the present disclosure is a method of treating aromatic L-amino acid decarboxylase (AADC) deficiency using adeno-associated virus (AAV)-AADC vector, comprising the following steps:

(a) Building viral vector of rAAV2-hAADC: A viral vector of serotype 2 of recombinant AAV (rAAV2)-human AADC (hAADC) is obtained, where the viral vector is built from hAADC between cytomegalovirus (CMV) promoter at beginning and SV40 poly A at end; where the viral vector has an adenovirus DNA structure including two inverted terminal repeats (ITR) at two ends of serotype 2 of AAV (AAV2); where the adenovirus DNA structure comprises CMV promoter, β-globin intron, hAADC and SV40 poly A sequentially between the two ITRs; and where the ITR has an antibiotic resistance gene at outboard.

(b) Injecting viral vector of rAAV2-hAADC: The viral vector of rAAV2-hAADC is directly injected into a brain by using a stereotactic technique for recovering activity of AADC in the brain.

The antibiotic resistance gene in step (a) is a resistant gene of ampicillin (Amp) or kanamycin (Kn).

The viral vector of rAAV2-hAADC is an unnatural gene obtained by a genetic engineering of restriction enzyme cleavage and by DNA ligation of β-globin intron, where the restriction enzyme cleavage is a cleavage of Cla I, EcoR V, Hind III, Not I, Sac II and Xho I in the AAV2 having ITRs at two ends.

Thus, a novel method of treating AADC deficiency using AAV-AADC vector is obtained.

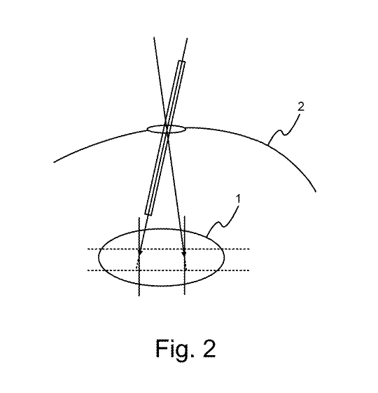

Please refer to FIG. 2, which is a view showing injection of a viral vector. As shown in the figure, a position for injection is found through a stereotactic technique. Holes on skull 2 near putamen 1 are drilled out and a long needle is used to inject 80 μl of rAAV2-hAADC into the putamen 1 at a velocity of 3 μl/min, where the rAAV2-hAADC has a density of 5×10¹¹ vg/ml and thus 1.6×10¹¹ vg of the viral vector is injected.

The AAV viral vector delivers AADC genes into putamens in striatums of three AADC deficiency patients. The first patient is a 5-year-old girl who had profound hypotonia and lack of any motor development. After the treatment according to the present disclosure, activities of the limbs are increased, control of the limbs is improved and recognition is enhanced. The girl can sit well without support with good head control and touch things with her hands one year after the treatment. The second patient is a 5-year-old boy. Two months after the treatment, activities of the limbs are obviously increased and control of the trunk is also improved. Activities of the limbs of the third patient are obviously increased one month after the treatment, too. Hence, the present disclosure uses the AAV viral vectors to transfer AADC genes for treating AADC deficiency practically and effectively.

The present disclosure directly injects a viral vector of rAAV2-hAADC into an AADC deficiency patient's brain through a stereotactic technique, which is an effective and novel treatment for AADC deficiency and, furthermore, is the only effective and (one of) the most successful human gene treatment until now. Thus, the present disclosure uses the state-of-the-art AAV treatment and deep brain stimulation to obtain an effective stereotactic technique for AADC deficiency treatment.

To sum up, the present disclosure is a method of treating AADC deficiency using AAV-AADC vector, where an AAV viral vector is used to transfer AADC gene by being directly injected into human brain through a stereotactic technique for solving the problem of movement disorder by recovering activity of AADV.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A method of treating aromatic L-amino acid decarboxylase (AADC) deficiency using adeno-associated virus (AAV)-AADC vector, comprising steps of:
(a) obtaining a viral vector of serotype 2 of recombinant AAV (rAAV2)-human AADC (hAADC),
wherein said viral vector is obtained from hAADC between cytomegalovirus (CMV) promoter at beginning and SV40 poly A at end;
wherein said viral vector has an adenovirus DNA structure;
wherein said adenovirus DNA structure includes two inverted terminal repeats (ITR) at two ends of serotype 2 of AAV (AAV2);
wherein said adenovirus DNA structure comprises CMV promoter, β-globin intron, hAADC and SV40 poly A sequentially between said two ITRs; and
wherein said ITR has an antibiotic resistance gene at outboard; and
(b) directly injecting said viral vector of rAAV2-hAADC into a brain by using a stereotactic technique.

2. The method according to claim 1,
wherein said antibiotic resistance gene is a resistant gene selected from a group consisting of ampicillin (Amp) and kanamycin (Kn).

3. The method according to claim 1,
wherein said viral vector of rAAV2-hAADC is an unnatural gene obtained by a genetic engineering of restriction enzyme cleavage and by DNA ligation of β-globin intron.

4. The method according to claim 1,
wherein said viral vector of rAAV2-hAADC is obtained by a restriction enzyme cleavage of Cla I, EcoR V, Hind Ill, Not I, Sac II and Xho I.


SEQUENCE LISTING (1 sequence)

SEQ ID NO: 1 - DNA, 5591 bp, Unknown
  AAV viral vector
tgacagatctgcgcgcgatcgatctgcgcgctcgctcgctcactgaggccgcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaggaattaattcgatatcaagctatccaacacactggtagggataacagggtaatctcgaggcaagcttgggcccggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagatcagcgctttaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatctttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcaca